(19) United States
(12) Patent Application Publication
(10) Pub. No.: US 2013/0268089 A1
(43) Pub. Date: Oct. 10, 2013

(54) ADAPTER FOR STOMACH DEVICES

(71) Applicants: Ram Weiss, Haifa (IL); Menachem P. Weiss, Haifa (IL)

(72) Inventors: Ram Weiss, Haifa (IL); Menachem P. Weiss, Haifa (IL)

(21) Appl. No.: 13/816,117

(22) PCT Filed: Aug. 9, 2011
(86) PCT No.: PCT/IB11/53550

§ 371 Date: Jun. 27, 2013

Related U.S. Application Data
(60) Provisional application No. 61/372,257, filed on Aug. 10, 2010.
(60) Provisional application No. 61/472,205, filed on May 2, 2011.

(30) Foreign Application Priority Data
Mar. 1, 2011 (GB) 1103467.5

(51) Int. Cl. A61F 2/04

(52) U.S. Cl. CPC A61F 2/04
USPC 623/23.68

(57) ABSTRACT

An inert adapter for supporting gastric devices. The adapter is connected to the inner lining of the stomach to encircle the esophageal sphincter and supports a treatment element. The treatment element is operated only after at least the healing period of the connection has passed. The adapter and the treatment element may be endoscopically inserted in a single action, and the activation of the treatment element may be carried out externally or internally by the dissolution of a dissolvable element. The adapter may support various treatment elements, such as an intake limiting pouch or an anti GERD device, and may additionally deliver drugs. Allowing a late activation of the device without additional intervention or even supervision simplifies gastric device applications and enhances their safety.

### BACKGROUND

1. Technical Field

The present invention relates to the field of gastric medical devices, and more particularly, to an endoscopic device connectable to the tissues inside the stomach.

2. Discussion of Related Art

Obesity is reaching epidemic proportions in the Western as well as the developing world. Along with the increase in the prevalence of simple obesity, there is a growing population of morbidly obese individuals (BMI>40 kg/m²) as well as super obese ones (BMI>50 kg/m²). As obesity carries increased risk of mortality and a broad spectrum of related co-morbidity, treatment approaches aimed at addressing this problem are needed. The conservative and most successful approach is of lifestyle modifications that include dietary changes and increased physical activity. The problem with this approach is the low compliance achieved and the limited benefit it provides for those with morbid and super obesity. There are several present and emerging pharmacological agents aimed at treating obesity yet their benefit seems to provide a modest and unsustainable weight loss, along with unpleasant side effects, and is diminished upon discontinuation of the drug.

The lack of success of conservative and pharmacological approaches to treat obesity lead to the emergence of bariatric surgery as the most effective and sustainable treatment option. Moreover, some bariatric surgical procedures result in hormonal changes that lead to improvement of conditions such as altered glucose metabolism and thus these procedures are also called “metabolic surgery”.

Bariatric procedures have two general mechanisms of action—restrictive and malapsorptive. The vast majority is performed using a laparoscopic approach and a laparotomy is rarely necessary. The simplest restrictive procedure is gastric banding in which an adjustable elastic band is used to create a small gastric pouch. The radius of the band can be changed using its subcutaneous bladder. Another procedure that is gaining popularity is the sleeve gastrectomy where a large portion of the stomach is resected leaving a narrow “sleeve” with limited volume. This procedure is claimed to have “metabolic” effects via reduction of ghrelin. The classic bariatric procedure is the Rouxen-Y gastric bypass (RYGB). In this procedure a small gastric pouch is created and anastomosed to a distal part of the small bowel while the stomach and duodenum are anastomosed distally. This leaves the proximal part of the small bowel without pancreatic enzymes and bile salts and reduces the area of absorption. Thus, the RYGB combines a restrictive and malapbsorptive component.

All bariatric procedures cause a greater weight loss than conservative approaches yet carry small but significant operative and peri-operative risks. This has lead to attempts to perform these procedures or imitate their effects via less invasive approaches. The simple approach to the gastro-intestinal tract is via the oral route, thus attempts at designing bariatric procedures that are performed using endoscopic machinery are actively pursued. The simplest one is the gastric balloon or other volume occupying devices that limit gastric capacity. These devices have been demonstrated to have a small short term effect on weight yet have significant side effects that have limited their use in clinical practice. Other approaches have attempted to suture the gastric wall using an endoscope thus reducing its volume and thus imitating the gastric sleeve procedure. Yet another approach contemplated a variable outlet that can be changed manually using a laparoscopic or endoscopic mechanism similar to the classic gastric band. Another attempt has been made to combine the restrictive and malabsorptive approach by connecting a flexible tube to the vicinity of the gastro-esophageal junction and leading it via the pylorus through the duodenum for a variable length. This approach creates a limited gastric pouch and adds a “bypass” component.

GERD is a common condition afflicting millions of adults and children that results from an anatomical or regulatory derangement of the gastro-esophageal sphincter that allows reflux of acid gastric contents into the esophagus. This condition is usually treated by pharmacological means and in severe case—by surgical means. There is currently no useful intra-gastric device to address this medical problem.

U.S. Pat. No. 7,037,344 discloses an artificial stoma device, a gastric sleeve device, an intestinal sleeve device and a combined gastrointestinal sleeve device. The following documents disclose various gastric pouches: U.S. Pat. Nos. 4,403,604, 6,981,978, 7,037,344, and 7,267,694, U.S. Patent Publication Nos. 20040122453, 20050267499, 2005240279, 20090012541, 2009093839, and 20100114130.

### BRIEF SUMMARY

Embodiments of the present invention provide a medical device comprising an inert tubular adapter comprising a proximal rim arranged to be connectable to an inner lining of the stomach to encircle the esophageal sphincter, the connection characterized by a specified healing period, and a treatment element affixable to the inert tubular adapter and having an operative state arranged to apply a treatment, wherein the medical device has an activated state in which the treatment element is affixed to the adapter and is in the operative state, and an inactive state in which the treatment element is not operative or is not affixed to the adapter, wherein the medical device is arranged to be activated only after a specified period that is equal to or longer than the healing period, to temporally separate the connection of the tubular adapter and the application of the treatment.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

The present invention will be more readily understood from the detailed description of embodiments thereof made in conjunction with the accompanying drawings of which:

FIGS. 1 to 7 are high level schematic illustrations of a medical device, according to some embodiments of the invention; and

FIG. 8 is a high level flowchart illustrating a gastric treatment method, according to some embodiments of the invention.

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

For a better understanding of the invention, the term “intake limiting pouch” in the present disclosure is defined in a non-limiting manner as an artificial mechanical structure (unlike common gastric pouches made at least in part from the stomach lining) connected within the stomach and encircling the esophageal sphincter, that receives the intake of food and liquids coming in through the esophageal sphincter, and at least partially holds the intake for a specified period of time, to limit the amount of possible intake in that time. The intake limiting pouch may hold and release the intake, hold and release a part of the intake, or create a mechanical barrier that postpones the transition of the intake from the esophagus to the stomach.

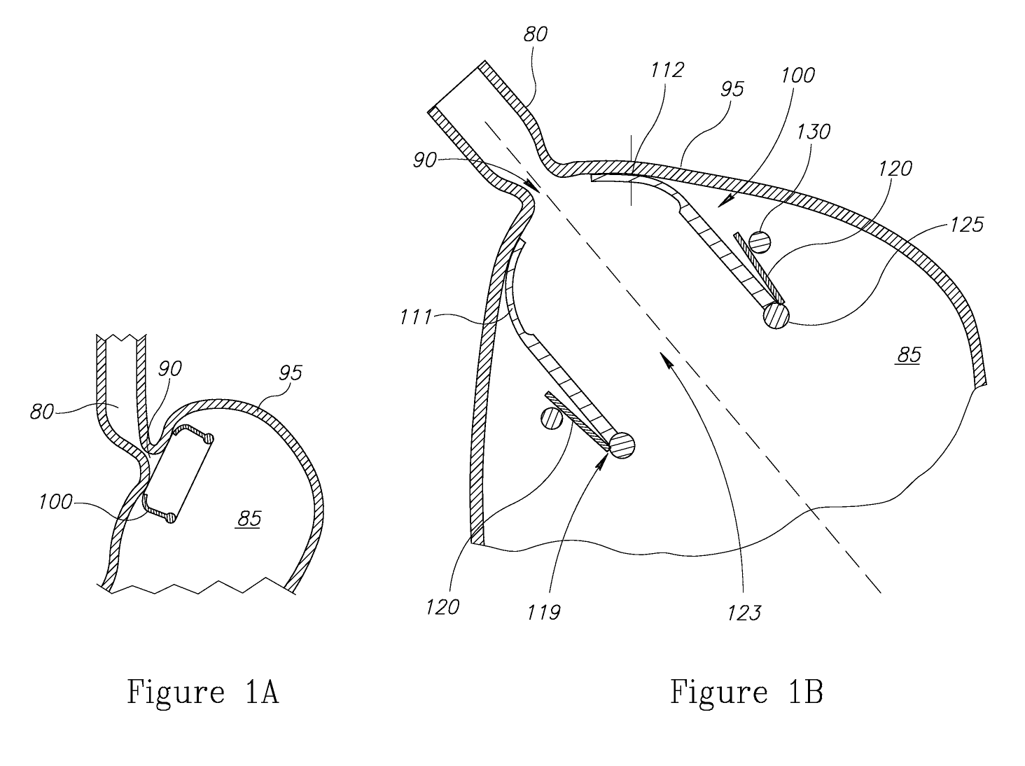

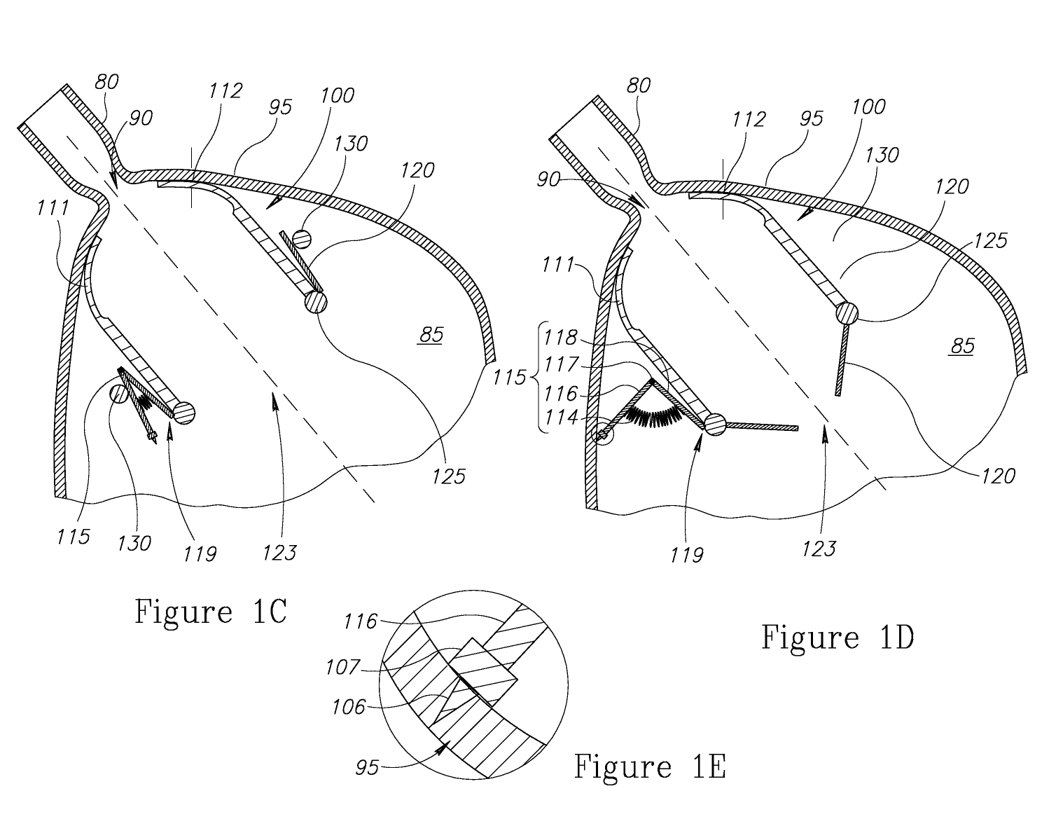

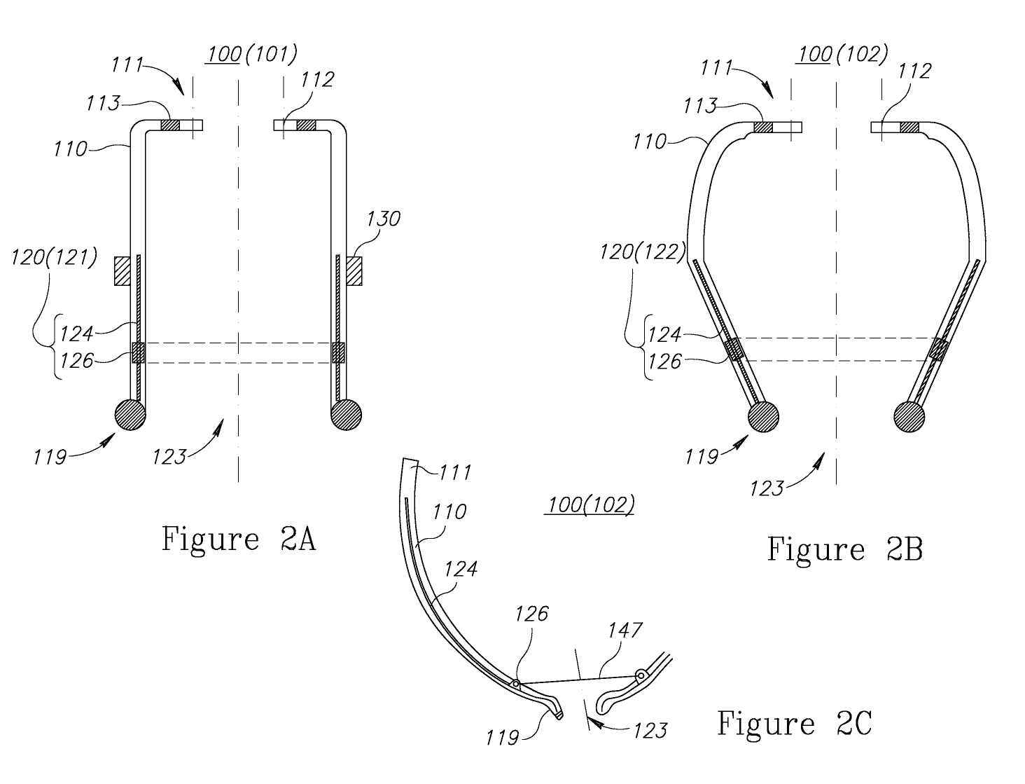

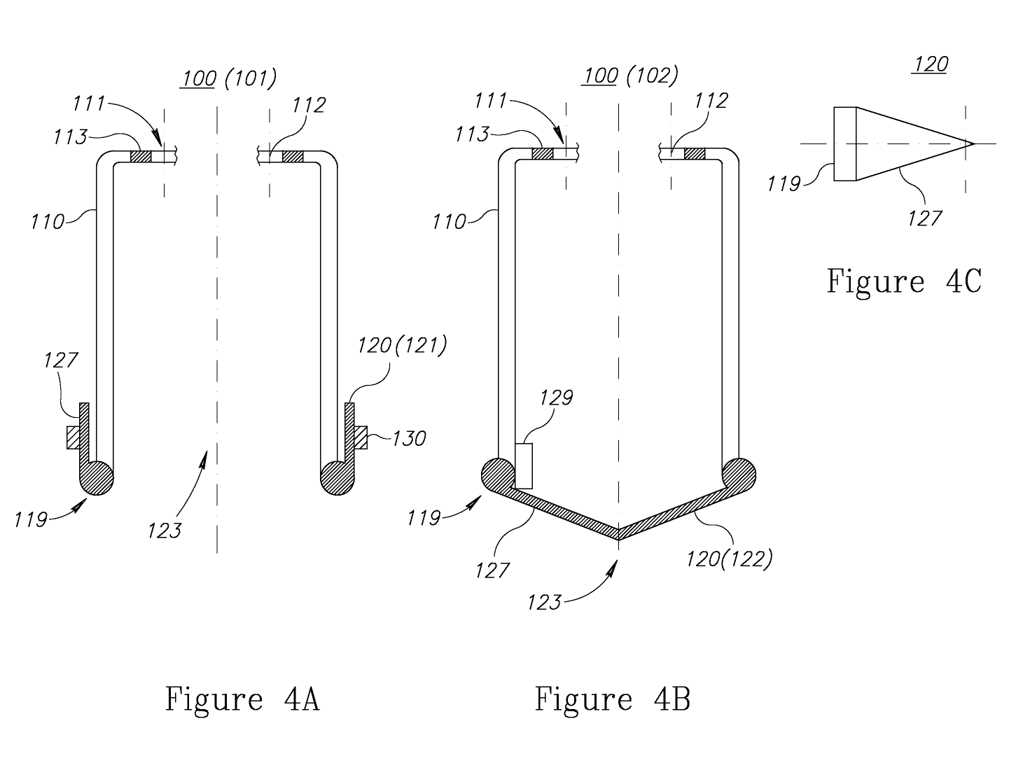

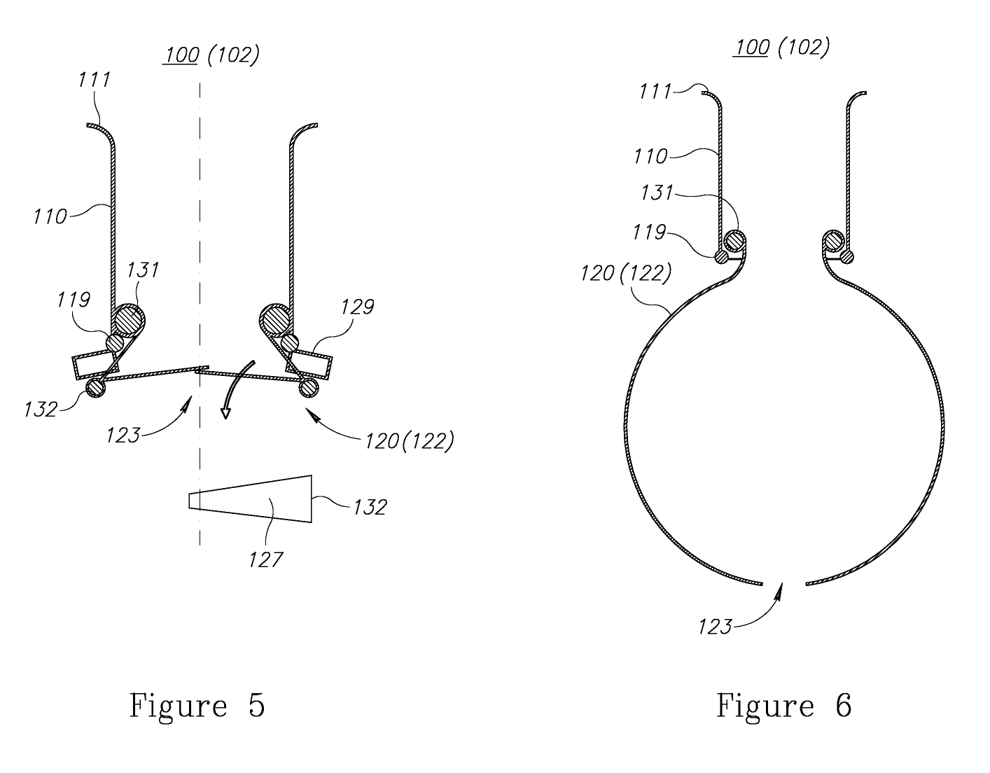

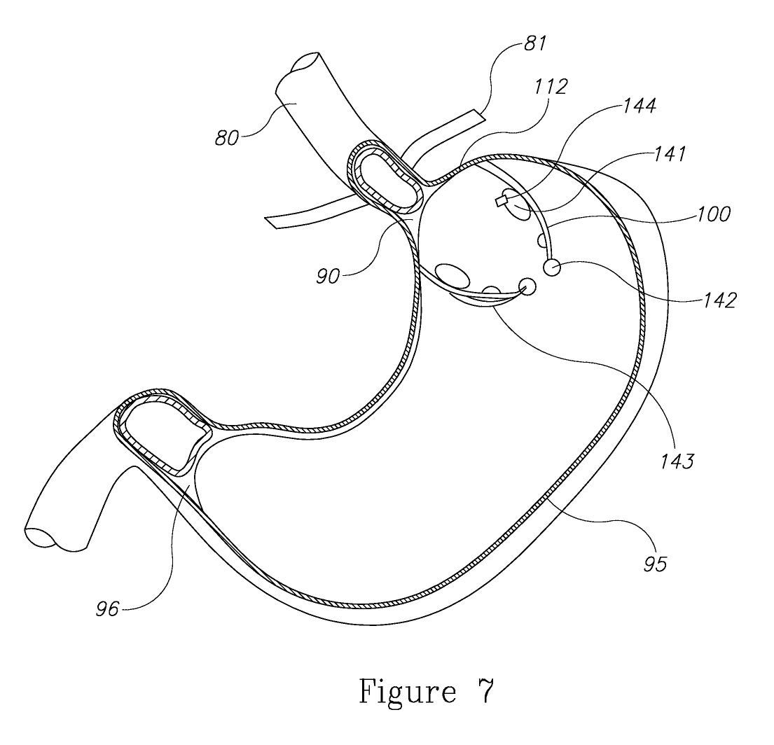

FIGS. 1 to 6 are high level schematic illustrations of a medical device 100, according to some embodiments of the invention. FIGS. 1A to 1E are cross sections of device 100 within the stomach 85, FIGS. 2 to 6 are cross sections of various configurations of device 100 in a two part configuration, and FIG. 7 illustrates device 100 as an intake limiting pouch in a single part configuration. FIGS. 2B, 2C, 3B, 6 and 7 illustrate device 100 operating as an intake limiting pouch, while FIGS. 4B and 5 illustrate device 100 operating as an anti GERD one way valve.

Medical device 100 comprises an inert tubular adapter 110 that is connected to the inner lining 95 of stomach 85, and a treatment element 120 (or parts thereof) that is affixable to inert tubular adapter 110. Treatment element 120 (or parts thereof) has an operative state 122 in which it is arranged to apply a treatment such as turning device 100 to an intake limiting pouch or to an anti-GERD one way valve, or to deliver a drug.

Medical device 100 has an inactive state 101, in which treatment element 120 is not operative or is not affixed to adapter 110, and an activated state 102, in which treatment element 120 is operative (122).

Tubular adapter 110 may have a diameter that is larger than the diameter of sphincter 90 in its maximal opening size, to allow some mechanical freedom to both device 100 and stomach lining 95.

Device 100 uses stomach lining 95 only as an anchoring area, and does not utilize lining 95 build the pouch or any parts of device 100.

Medical device 100 is arranged to be activated only a specified period after the connection of tubular adapter 110 to inner lining 95 of stomach 85. The specified period is equal to or longer than the healing period of the connection itself, to separate temporally the connection of tubular adapter 110 and the application of the treatment.

For example, medical device 100 may be assembled prior to its insertion to stomach 85, inserted and attached endoscopically to inner lining 95, and have a delayed activation mechanism. Alternatively, only adapter 110 may be inserted and attached endoscopically to inner lining 95, and after the specified period, only treatment element 120 may be inserted endoscopically and attached to adapter 110. Treatment element 120 may be operative (122) directly after its attachment to adapter 110, or activation of device 100 may be further delayed by a holding element 130, as described below. Device 100 may be self activated by a mechanical or chemical delayed release mechanism, or activated externally by a signal of any kind. Additionally, treatment element 120 may be replaced endoscopically after or even before a first use upon corresponding indications.

In embodiments, inert tubular adapter 100 may be connected to stomach 85 at an occurring opportunity and stay inert and inactivated for a long period within stomach 85. Treatment element 120 may be operated (122) or connected to adapter 110 at any later occasion.

In contrast to prior art methods and devices, medical device 100 temporally separates the attachment phase from the beginning of treatment, and ensures thereby both proper healing of attachment area 112 before the commencement of the treatment as well as the safety of the associated force application.

During the healing period the patient is not limited in food consumption as the inner passage of the adapter is larger than esophageal sphincter 90. In this way, force application on connection area 112 (sutures or other means of connection) is avoided, and good healing of attachment area 112 is achieved. Hence, in contrast to prior art methods and devices which require supervision and restrictions to food consumption during the healing period, patients treated in the proposed procedures are not limited in regards to food consumption during the healing period. Furthermore, the treating physicians have a better control on the treatment, as device 100 allows a better control on the commencement of the treatment, and also allows temporary or permanent cessation of the treatment by changing the state of treatment element 120 to inoperative, or removing treatment element 120 altogether, without wounding or damaging stomach 85, as adapter 110 may be left connected in stomach 85. Exchanging treatment element 120 may also be carried out endoscopically, without damaging the connection of adapter 110 to stomach 85. Any or both adapter 110 and treatment element 120 may be removed from stomach 85 by a single procedure.

Inert tubular adapter 110 comprises a proximal rim 111 and a distal rim 119. Proximal rim 111 is arranged to be connectable to inner lining 95 of stomach 85, to encircle the esophageal sphincter 90, such that the fluid connection of stomach 85 with esophagus 80 is carried out via device 100.

Proximal rim 111 may be arranged to enable its connection (112) to inner lining 95 of stomach 85 by suturing, gluing, clipping, stapling, and riveting. Riveting may comprise using blind rivets with expanding large caps on the opposite side of the insertion side. Riveting may utilize non-blind rivets. The rivets may be connected to each other on the serosal side of the tissue.

The whole or part of medical device 100 may have an inactive state 101 and an activated state 102, for example a deformation of tubular adapter 110 may participate in the activation of device 100.

Treatment element 120 is affixable to distal rim 119 and has operative state 122 arranged to apply a treatment. Operative state 122 may comprise a mechanical deformation, a position change, a change in chemical character or any other change that affects the stomach or the food passing through device 100.

Tubular adapter 110 may be arranged to receive treatment element 120 after the specified healing period has passed. For example (FIGS. 1B, 3A and 4A), distal rim 119 may comprise a flange 125 arranged to engage and affix treatment element 120 that is inserted after the healing period. The insertion of treatment element 120 does not involve any tissue injury and its connection to the already established tubular adapter 110 is purely mechanical. This insertion stage may also be use to control the healing and ensure the safe onset of the treatment.

In embodiments, treatment element 120 may be affixed to the adapter walls or be embedded within the walls (see below, FIGS. 1B, 2A, and 2B). In inactive state 101 of device 100, treatment element 120 may be at least partially inserted within a body of inert tubular adapter 100, and in active state 102 of device 120, treatment element 120 may either stay embedded within adapter 110 causing it to mechanically deform, or may be at least partially released from the adapter body to assume operative state 122.

Tubular adapter 110 and treatment element 120 may be arranged to be endoscopically inserted into stomach 85 by a single insertion procedure, with treatment element 120 in an inoperative state 121. A transition of treatment element 120 from inoperative state 121 to operative state 122 may be carried out upon a specified condition that takes place after the specified healing period.

Medical device 100 may comprise at least one holding element 130 such as dissolvable elements arranged to change the state of treatment element 120 from inoperative 121 to operative 122, or the state of device 100 from inactive 101 to active 102. The specified condition may be a dissolution of the dissolvable element(s), upon which the transition of treatment element 120 to operative state 122 occurs by a configurational change of medical device 100. The dissolvable elements may comprise a band, a pin or a thread.

Holding element 130 may have a toroidal shape, e.g. be a ring or a band, flexible or stiff or made of surgical suture. Holding element 130 may be arranged to release parts of treatment element 120 by dissolution of dissolvable elements in holding element 130. Holding element 130 may be made of plastic, metal, fabric, shape memory material and their combinations.

Holding element 130 may be associated with the activator that receives an external signal and operates treatment element 120 by cutting or dissolving holding element 130.

For example (FIG. 1B), holding element 130 may comprise a dissolvable ring or a set of dissolvable rings that are arranged to hold treatment element 120 in inoperative state 121, and upon dissolution release treatment element 120 to take on operative state 122. Treatment element 120 may comprise flaps connected to distal rim 119 and held against tubular adapter 110 either externally—embedded in tubular adapter 110 (left side of device 100 in FIG. 1B) or external to tubular adapter 110 (right side of device 100 in FIG. 1B) by rings 130.

The specified condition may comprise an external signal (e.g. electromagnetic radiation, ultrasound signal, magnetic or electric signal). Treatment element 120 may comprise an activator (not shown) arranged to receive the external signal and activate device 100.

The specified condition may comprise an internal or an external stimulus of physical, mechanical, chemical electric or thermal nature. For example, the stimulus may comprise temperature, and the activation of device 100 may be carried out by drinking a hot fluid. In another example, activation of device 100 may be carried out by drinking a fluid having certain chemical character, such as reactivity with holding element 130, induction of a certain pH level to dissolve holding element 130 etc.

The specified condition may comprise a pre-determined self activation, an exogenously induced activation (such as an external signal detected by an activator, or an induced activation by an administered substance causing e.g. a chemical reaction, a temperature change or an energy supply to device 100) and an endoscopically performed activity (such as cutting holding element 130 to release treatment element 120).

The specified condition may be incorporated into holding element 130 as a chemical or mechanical character that causes activation of device 100 after a specified period. In this case the specified condition is inherent in the structure or composition of holding element 130 or device 100.

Dissolvable element as holding element 130 may be made of PLGA (poly (lactic-co-glycolic acid)), PGA (Polyglycolic acid), Caprolactones and any other known biocompatible and bio-dissolvable materials. The dissolution period of dissolvable element 130, that may partly or fully determine the activation period of device 100, may result from dissolution characteristics of the materials involved or from interaction of these materials with external signals, an internal or external stimulus, or intake liquids. For example, a patient may drink a liquid that promotes the dissolution of dissolvable elements 130, like having chelators that scavenge divalent cations to break dissolvable elements 130, or simply a hot fluid that promotes dissolution of dissolvable elements 130. In embodiments, a mechanical intervention may be used to activate device 100, for example cutting or releasing holding element 130 endoscopically.

To make it more flexible at insertion to stomach 85, holding element 130 may be a flexible tape or yarn made of dissolvable materials. When these materials dissolve at the end of the healing period, e.g. in a week or more, device 100 changes to activated state 102, e.g. taking on a final shape, and performs its therapeutic activity.

Treatment element 120 may comprise flaps that are positioned within tubular adapter 110. Upon activation, device 100 may change it form to constrict the passage of food to the stomach.

Tubular adapter 110 may be made of at least one biocompatible semi-permeable flexible material (e.g. rubber, plastic, metal, carbon and other fibers, or any combination thereof) or non-permeable materials. The structure of tubular adapter 110 may be mesh-like, membranous, or fibrous at different part of adapter 110 or in adapter 110 as a whole. Tubular adapter 110 may have a longitudinally variable flexibility, selected to allow the connection of proximal rim 111 to inner lining 95, to stabilize device 100, to allow a change in a spatial configuration of device 100 upon its transition from inactive 101 to activated 102 state, and to provide a distal support of treatment element 120.

In embodiments of device 100 as an anti-GERD device, such as a one way valve, adapter 110 and treatment element 120 may be made of impermeable material.

The mechanical strength of tubular adapter 110 may change longitudinally (e.g. FIGS. 2A, 2B, 2C) to generate a mechanical change of structure of device 100, to perform the treatment (e.g. constriction of food passage). The flexibility and strength of tubular adapter 110 may change in different regions to support the transition from inactive state 101 to activated state 102 of device 100 and to support its functionality.

Tubular adapter 110 may have a longitudinally variable permeability selected to control fluid exchange between the stomach lumen and an internal volume of adapter 100.

Tubular adapter 110 may comprise a rigid area 113 near proximal rim 111 that attenuates forces acting through tubular adapter 110 on attachment area 112. Rigid area 113 may be ring shaped and arranged to protect attachment area 112 from forces that could be activated by tension in tubular adapter 110 in its activated state 102, e.g. operating as an intake limiting pouch. Area 113 can be made rigid in tension but flexible enough to enable insertion of device 100 via the esophagus.

Treatment element 120 may comprise at least one supportive element 124 that is embedded within a more flexible distal part of tubular adapter 110 (FIGS. 2A, 2B). Supportive elements 124 may be rigid or flexible, and have varying thickness.

Supportive elements 124 may comprise rigid beams embedded longitudinally in tubular adapter 110, or one or more rigid tubular elements. The rigid beams may be pivoted on at least one supportive element 126 embedded in tubular adapter 100 (FIGS. 2A, 2B, 2C) and are arranged, in operative state 122 of treatment element 120, to distally constrict distal orifice 123 of device 100 upon proximal pressure applied by food internally on adapter 110.

The distal ends of supportive elements 124 (e.g. rigid thin beams) may be interconnected by an annular element 147, such as a thread yarn or an annular spring, arranged to define a size of distal orifice 123 and to allow an endoscopic manual manipulation of a size of distal orifice 123. Distal orifice 123 may have a constant or an adjustable size. In particular, as illustrated below, a structure of the intake limiting pouch may be arranged to decrease a size of distal orifice 123 as a function of an increasing filled volume of the pouch, and vice versa. Either device 100 as a whole (adapter 110 and treatment element 120) or treatment element 120 alone may function as the intake limiting pouch.

Annular element 147 may function as supportive element 126—to urge supportive elements 124 into place to change the spatial configuration of device 100, e.g. to take the form of an intake limiting pouch in activated state 102. Annular element 147 may have this function in device 100 either in a two part configuration (e.g. FIGS. 1B, 6) or in one part configuration (FIG. 7).

Supportive element 126 may comprise a rigid ring arranged to urge supportive elements 124 into place. Holding element 130 may be arranged to restrain supportive elements 124 (e.g. the rigid beams) in inactive state 101 of device 100, and to release supportive elements 124 to deform device 100 to a form of the intake limiting pouch, or any other active form. Pivot 126 may comprise a rigid ring that urges the rigid beams to deform device 100 into the activated formation.

Supportive elements 124 may be embedded in tubular adapter 110 with a mechanical tendency to bend device 100 inwards, i.e. to change from inoperative state 121 (FIG. 2A) to operative state 122 (FIGS. 2B, 2C). Supportive elements 124 may be pressed to retain inoperative state 121 by biodissolvable ring 130.

Treatment element 120 may further comprise a pivot as supportive element 126 connected to supportive elements 124 and arrange to support a turning movement of supportive elements 124 in respect to pivot 126, to generate a constriction of distal rim 119, controlling the size of orifice 123. Due to the constriction, device 100 takes, in activated state 102 a form of an intake limiting pouch.

Furthermore, the proximal end of at least one supportive element 124 may be effected by the content of tubular adapter 110. For example, as the pouch formed in activated state 102 of device 100 gets fuller, supportive element 124 may increase their turning angle around pivot 126 and so decrease the size of orifice 123 to generate a larger resistance to food movement through device 100 and thereby a feeling of satiation. As a result, when ring 130 dissolves, device 100 turns into an intake limiting pouch with a self-regulating outlet 123.

Outlet orifice 123 that limits the evacuation of food from the pouch into the stomach, may be of fixed size, or be self adjusting that such as to change its outlet area as a function of the fullness of the pouch, namely the fuller the pouch the smaller the exit area and vice-versa—when the pouch is empty the exit area may be larger, back to its original size.

Supportive elements 124 may have a variable elasticity, for example a higher elasticity on their distal end to allow a large range of sizes for orifice 123. Distal end 119 of tubular adapter may have a corresponding elasticity.

In embodiments of device 100 as an intake limiting pouch with distal orifice 123, the size of orifice 123 may be either fixed or decreasing by an increasing filled volume of the pouch. Orifice 123 may be adjustable and /or periodically openable according to specified criteria. At least one supportive element 124 may be flexible and comprise a thread yarn that may be tightened endoscopically if needed to regulate the size of orifice 123. Treatment element 120 may comprise a thread yarn embedded around distal orifice 123 and arranged to allow an endoscopic manual manipulation of a size of distal orifice 123. Treatment element 120 may comprise a distal circumferential spring, arranged to constrict distal orifice 123.

In embodiments, tubular adapter 110 may maintain a cylindrical shape in both inactive 101 and activated 102 states of device 100, while treatment element 120 forms alone the constriction of distal opening or orifice 123 of the formed intake limiting pouch.

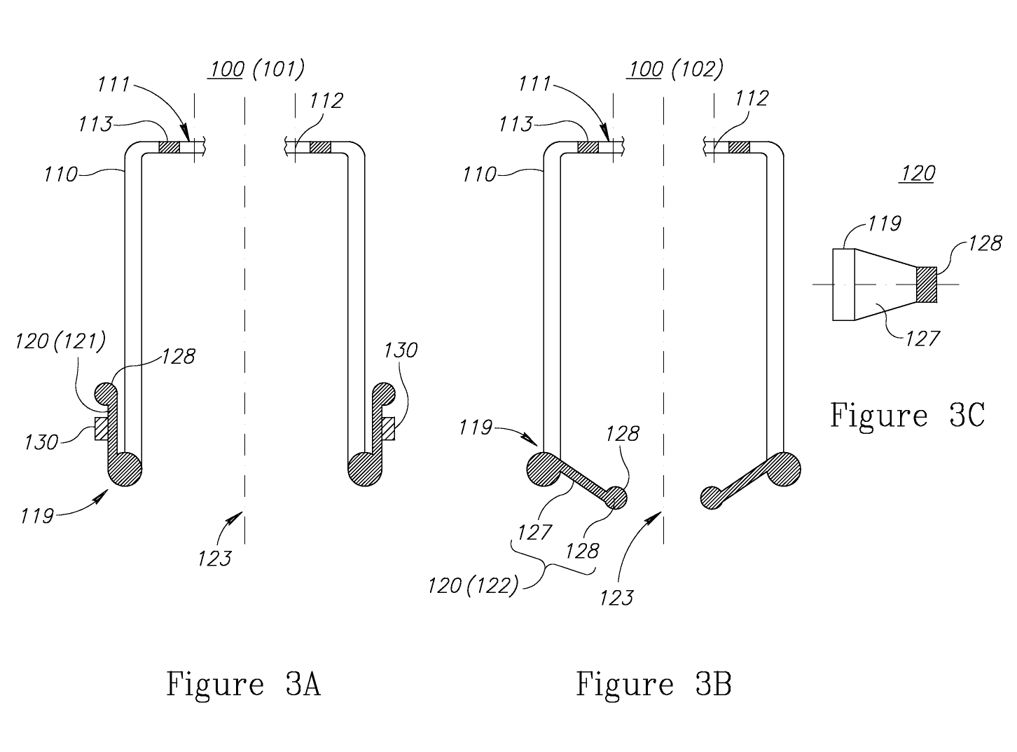

For example (FIGS. 3A, 3B), treatment element 120 may comprise flaps 127 arranged to be held by dissolvable ring 130 in inoperative state 121 and released in operative state 122, with distal rim 119 serving as a pivot. Flaps 127 may be trapezoid (FIGS. 3C) and have rounded edges 128 that define orifice 123. Flaps 127 may be held in place by the elasticity of distal rim 119 (distal rim 119 may be pre-tensioned) or by attached elements (not shown).

In embodiments, treatment element 120 may form a one way valve arranged to prevent stomach fluid from reaching esophagus 80 (FIGS. 4A, 4B). Flaps 127 may be triangular (FIGS. 4C) and may in operative state 122 close orifice 123 partially or completely. Flaps 127 may be held in activated state 102 of device 100 such as to allow only downwards movement (into stomach 85) of food from esophagus 80 through device 100, and prevent backward movement—up into device 100 and esophagus 80. For example, flaps 127 may be stopped by detents 129 from folding away from closed orifice 123 upon upward pressure of stomach fluids. Flaps 127 may move downwards, against a spring force (e.g. exerted by distal rim 119 or an additional spring element) to let food enter stomach 85 smoothly, but flaps 127 may be disabled from moving upward beyond the fully closed shape when no pressure is activated from esophagus 80 into stomach 85. Device 100 thus functions as an anti GERD (Gastroesophageal reflux disease) element that lets food exit from device 100 but does not let stomach juices or any other elements enter device 100. Flaps 127 may be held in inoperative state 121 by holding elements 130. In this case device 100 is made of non permeable materials.

FIG. 5 illustrates another embodiment of device 100 as an anti GERD element, an embodiment in which flaps 127 are folded internally within the distal end of tubular adapter 110. Like the embodiment in FIG. 4B, detents 129 prevent flaps 127 from folding in the direction of esophagus 80, thereby blocking stomach contact from entering device 100. In the illustrated embodiment flaps 127 are trapezoidal, and close orifice 123 completely with some overlap that stabilizes the valve they create. The illustrated configuration comprises an attachment rim 131 that interlocks with distal rim 119 of tubular adapter 119 and affixes treatment element 120 thereto, as well as a pivot 132 for enabling movement of flaps 127 to allow food enter stomach 85.

Treatment element 120 may be an intake limiting pouch (FIG. 6) with distal orifice 123 having a size that is either fixed or decreasing by an increasing filled volume of the pouch. Treatment element 120 as the intake limiting pouch may have a sealable distal orifice 123 that is periodically opened according to specified criteria, to allow passage of food.

Treatment element 120 may be attached to tubular adapter 110 either during the insertion of tubular adapter 110 or later, e.g. after the healing has ended. The attachment of treatment element 120 as an intake limiting pouch to tubular adapter 110 may be carried out by rim 131 of treatment element 120 pressed into and held by flange 125 associated with distal rim 119 or by rim 119 itself. Rim 131 of treatment element 120 may be, for example, a toroidal flange, a bayonet, a screw types, a band type, or any flexible connection.

Treatment element 120 may comprise a distal circumferential spring (not shown), arranged to constrict distal orifice 123 of device 100.

Medical device 100 may further comprise a drug delivering element 115 (FIGS. 1C, 1D) arranged to support the healing and/or the treatment. Drug delivering element 115 may be arranged to deliver a drug into any of: stomach 85 lumen, stomach lining 95 (e.g. attachment area 112), or a vicinity of esophagus 80. For example, drug delivering element 115 may comprise a spring (114) loaded delivering element 116 arranged to pierce stomach lining 95 to deliver the drug. Delivering element 116 may have a sharp edge 106 surrounded by a stopping area 107 (FIG. 1E) that prevents excessive penetration into stomach lining 95 as a result of peristaltic waves. Delivering element 116 may be pivoted at pivot 117 on a basal element 118. Drug delivering element 115 may be restrained by holding element 130 and be released together with treatment element 120. Alternatively, drug delivering element 115 may be restrained by an additional holding element 130, and released before or after the release of treatment element 120.

In embodiments (FIGS. 4B, 5), flaps 127 may be arranged to hold food or fluids within device 100 for a specified period before releasing it into stomach 85. For this use flaps 127 may be reinforced and associated with a timing mechanism.

Treatment element 120 may be selected to be attached on a connected adapter 110 at any time after the specified period has passed. Moreover, treatment element 120 may be replaced by with another treatment element 120 (to apply a different or a modified treatment, or to renew an effective treatment element 120) while maintaining adapter 110 connected to stomach 85.

Inert tubular adapter 110 and treatment element 120 may be integrated, and device 100 may be functional in a single part configuration as an intake limiting pouch, as illustrated in FIGS. 7 and 2C. Device 100 is connected proximally (111) at a connection area 112 to stomach 85 in around esophageal sphincter 90 (located below diaphragm 81). Rim 119 of device 100 may comprise an outlet ring 142 that is toroidal and hollow.

One or more fluid containers 141 (FIG. 7) may be attached to the inner lining of device 100 or be embedded in the wall of device 100. Containers 141 may be connected to outlet ring 142 by one or more flow channels 143, such as to fill hollow ring 142 under control of an inflating/deflating valve 144 connected to containers 141. When the pouch fills with food, pressure is generated on one or more containers 141, and the pressure passes into hollow ring 142 which inflates and reduces outlet exit 123. When the food finally leaves the pouch, the pressure diminishes and the outlet area shrinks back to its initial size. The baseline size of outlet 123 may be determined by setting the initial pressure within device 100. For example, valve 144, that may be located within the pouch may be arranged to enable change of the initial pressure via endoscopic means. This embodiment may be implemented in device 100 either in its one part or in its two part configuration.

In another embodiment (FIG. 2C), thin and narrow beams 124 are embedded within the pouch or attached to the pouch. When the pouch expands as it fills with food, beams 124 move or bend to the outside direction with the pouch body, but because the lower, distal part is kept in constant perimeter by a limiting structural element as supportive element 126, that is connected distally to device 100 the outlet exit area 123 is reduced by the lower parts of thin beams 124. Limiting structural element 126 may be enforced by reinforcement 147, such as a cord, a spring, or other means. Other possibilities for implementing reinforcement 147 are mechanical hinges or shutters that can be moved to enable the insertion of device 100 via esophagus 80 (see for example FIGS. 3A and 3B). Limiting structural element 126 or reinforcement 147 may comprise a round or flat metal spring attached to the pouch, embedded in it or threaded into the holes in bulges that are part of the pouch. The spring may be inserted into the stomach having a small diameter, and expanded to a larger diameter after insertion, and locked in the opened state. The larger diameter in the opened state may be selected to eliminate the possibility of a free pouch to pass via pylorus 96, typically larger than 25 mm The area of outlet 123 is regulated by pouch volume. A drain tube may be attached to the pouch, near outlet 123 and drain all or part of the food from pouch to pylorus 96 or to distal parts of stomach 85. The tube can be an inherent part of the pouch, alternatively it can be attached permanently, or attached with a detachable connection. The attachment is flexible and does not hinder the variation of outlet area 123. The tube may lead through stomach 85, through pylorus 96 and down below the duodenum. In this embodiment the food passes through the drain tube directly to the small intestine.

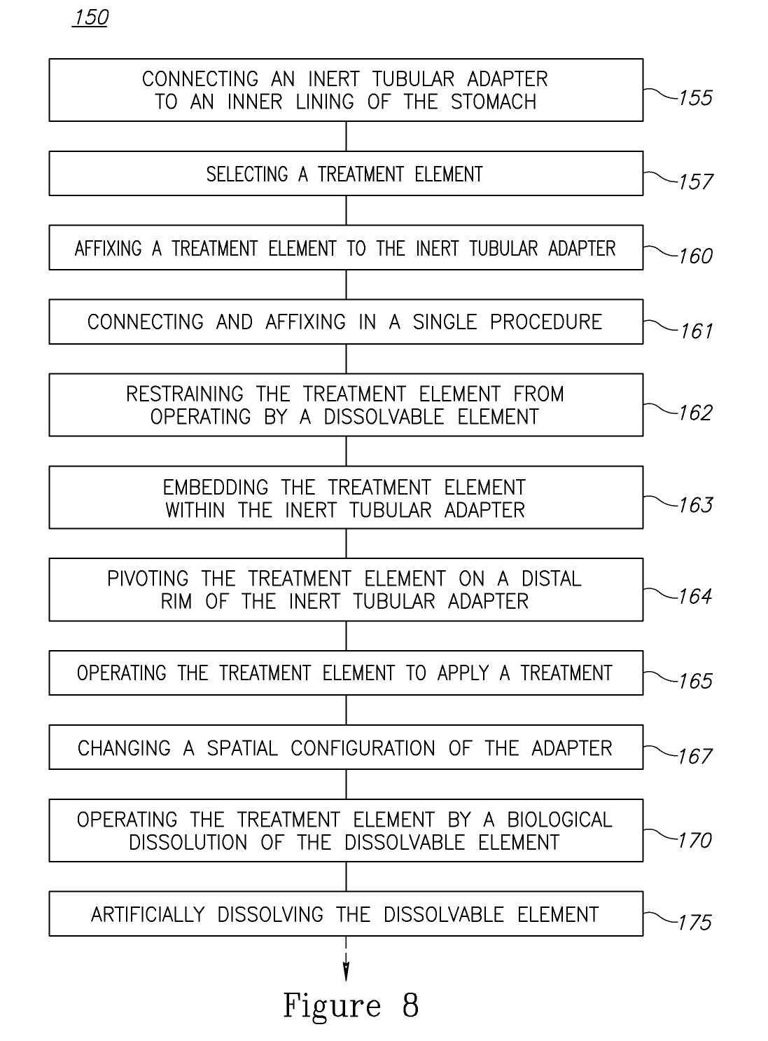

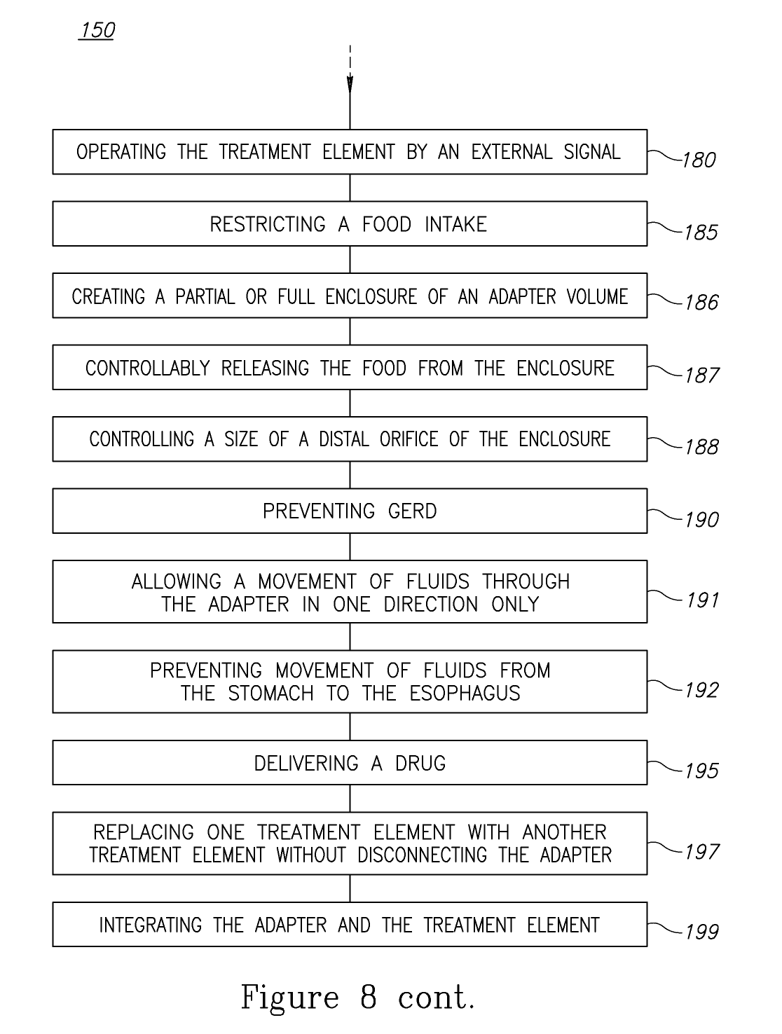

FIG. 8 is a high level flowchart illustrating a gastric treatment method 150, according to some embodiments of the invention.

Method 150 comprises the following stages: connecting an inert tubular adapter to an inner lining of the stomach (stage 155) to encircle the esophageal sphincter, the connection characterized by a specified healing period, affixing a treatment element to the inert tubular adapter (stage 160), and operating the treatment element to apply a treatment (stage 165), wherein the operating (stage 165) is carried out a specified period after the connecting (stage 155), the specified period being equal to or longer than the healing period, to temporally separate the connection of the tubular adapter (stage 155) and the application of the treatment (stage 165).

Method 150 may comprise restricting a food intake (stage 185), wherein the treatment is creating a partial or full enclosure of an adapter volume (stage 186), and controllably releasing the food from the enclosure (stage 187).

Method 150 may comprise preventing GERD (stage 190), wherein the treatment is allowing a movement of fluids through the adapter in one direction only (stage 191), namely from the esophagus to the stomach and not in the opposite direction (stage 192).

Method 150 may further comprise delivering a drug (stage 195) to the surroundings of the adapter.

Connecting (stage 155) and affixing (stage 160) may be carried out in a single procedure, and operating (stage 165) may be carried out later, either by a natural (stage 170) or artificial (stage 175) dissolution of a dissolvable element that restrains the treatment element from operating when affixed to the adapter (stage 162), or by an external signal (stage 180). Alternatively, affixing (stage 160) may be carried out in a separate procedure from connecting (stage 155).

Affixing (stage 160) may comprise at least partially embedding the treatment element within the inert tubular adapter (163), or pivoting the treatment element on a distal rim of the inert tubular adapter (164).

Operating (stage 165) may comprise changing a spatial configuration of the adapter (stage 167), e.g. to cause the adapter to take on a pouch form. Method 150 may further comprise controlling a size of a distal orifice of the pouch (stage 188), externally or mechanically according to a degree of fullness of the pouch.

Method 150 may further comprise integrating the adapter and the treatment element to a single device (stage 199).

Method 150 may further comprise selecting a treatment element (stage 157), e.g. as an intake limiting pouch, an anti-GERD device, a drug delivering device etc. according to the clinical status of the patient. Method 150 may further comprise replacing one treatment element with another treatment element (stage 197), while maintaining the adapter connected to the stomach.

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention.

1. A medical device comprising:
an inert tubular adapter comprising a proximal rim arranged to be connectable to an inner lining of the stomach to encircle the esophageal sphincter, the connection characterized by a predefined healing period, and
a treatment element affixed to the inert tubular adapter and having an operative state arranged to apply a treatment and an inactive state in which the treatment element is not operative,
wherein:
the tubular adapter and the treatment element are arranged to be endoscopically inserted into the stomach by a single insertion procedure, with the treatment element in the inactive state, and
the treatment element is arranged to be self-activated by a delayed release mechanism after a specified period that is equal to or longer than the predefined healing period and upon a specified condition, to temporally separate the connection of the tubular adapter and the application of the treatment.

2. The medical device of claim 1, wherein the specified condition is at least one of: a pre-determined self activation, an exogenously induced activation and an endogenously induced activation.

3-5. (canceled)

6. The medical device of claim 1, further comprising an activator arranged to receive an external signal as the specified condition and to activate the treatment element thereupon, wherein the external signal is at least one of: an electrical signal, a magnetic signal and an acoustic signal.

7. (canceled)

8. The medical device of claim 1, wherein the specified condition is induced activation by an administered substance causing at least one of: a chemical reaction with the medical device, a temperature change of the medical device, and an energy supply to the medical device.

9. The medical device of claim 1, further comprising at least one holding element arranged to restrain the treating element in the inactive, and to release the treatment element, upon the specified condition, to the operative state to activate the device.

10. The medical device of claim 9, wherein the at least one holding element is toroidal, surrounds the adapter and the treatment element, comprises at least one of: a ring; a band; and a thread, and is made of at least one of: plastic, metal, fabric and shape memory material.

11. The medical device of claim 9, wherein the at least one holding element comprises at least one dissolvable element, wherein the self activation is a dissolution of the at least one dissolvable element, upon which the transition of the treatment element to the operative state changes a spatial configuration of the medical device, wherein the at least one dissolvable element comprises at least one of: a ring; a band; a pin and a thread.

12-13. (canceled)

14. The medical device of claim 11, wherein the at least one dissolvable element is at least one dissolvable ring or band or thread yarn, and wherein the treatment element is connected to the distal rim and held in its inactive state by the dissolvable ring or band or thread yarn.

15. (canceled)

16. The medical device of claim 1, wherein the tubular adapter has a longitudinally variable permeability selected to control fluid exchange between the stomach lumen and an internal volume of the adapter.

17-18. (canceled)

19. The medical device of claim 1, wherein the treatment element is a one way valve arranged to prevent stomach fluid from reaching the esophagus, wherein the one way valve comprises reinforced flaps, arranged to hold content within the device for a specified period before releasing the content into the stomach.

20-21. (canceled)

22. The medical device of claim 1, wherein at least one of: the treatment element, and the medical device as a whole, is an intake limiting pouch with a distal orifice having an adjustable size.

23-24. (canceled)

25. The medical device of claim 1, wherein the treatment element is a one way valve arranged to prevent stomach fluid from reaching the esophagus and wherein a structure of the intake limiting pouch is arranged to decrease a size of the distal orifice as a function of an increasing filled volume of the pouch, and vice versa.

26. The medical device of claim 1, wherein the treatment element is a one way valve arranged to prevent stomach fluid from reaching the esophagus and wherein the treatment element comprises a plurality of rigid beams that are pivoted on at least one supportive element embedded in the tubular adapter and are arranged, in the operative state of the treatment element, to constrict the distal orifice as a function of a pressure applied by food internally on the adapter.

27. The medical device of claim 26, further comprising a holding element arranged to restrain the rigid beams in the inactive state of the device, and to release the rigid beams to deform the device to a form of the intake limiting pouch.

28. The medical device of claim 26, wherein the at least one supportive element comprises an annular element arranged to define a size of the distal orifice, wherein the annular element is a tied thread yarn or an annular spring arranged to allow an endoscopic manual manipulation of the size of the distal orifice.

29-32. (canceled)

33. The medical device of claim 1, wherein the proximal rim is arranged to enable its connection to the inner lining of the stomach by at least one of: suturing, gluing, clipping, stapling, and riveting.

34-37. (canceled)

38. A kit comprising:
an inert tubular adapter comprising a proximal rim arranged to be connectable to an inner lining of the stomach to encircle the esophageal sphincter, the connection characterized by a predefined healing period, and
at least one treatment element affixed to the inert tubular adapter and having an operative state arranged to apply a treatment and an inactive state in which the treatment element is not operative,
each packed in a separate sterile package,
wherein:
the tubular adapter and the treatment element are arranged to be endoscopically inserted into the stomach by a single insertion procedure, with the treatment element in the inactive state, and
the treatment element is arranged to be self-activated by a delayed release mechanism after a specified period that is equal to or longer than the predefined healing period and upon a specified condition, to temporally separate the connection of the tubular adapter and the application of the treatment.

39. A method comprising:
connecting an inert tubular adapter to an inner lining of the stomach to encircle the esophageal sphincter, the connection characterized by a specified healing period;
affixing a treatment element to the inert tubular adapter; and
operating the treatment element to apply a treatment,
wherein the operating is carried out a specified period after the connecting, the specified period being equal to or longer than the healing period, to temporally separate the connection of the tubular adapter and the application of the treatment.

40. The method of claim 38, wherein the connecting and the affixing are carried out in a single procedure, and the operating is carried out later by dissolving a dissolvable element that is arranged to restrain the treatment element from operating when affixed to the adapter.

41. The method of claim 38, further comprising replacing one treatment element with another treatment element, while maintaining the adapter connected to the stomach.
